(19) United States
(12) Patent Application Publication
(10) Pub. No.: US 2025/0352745 A1
(43) Pub. Date: Nov. 20, 2025

(54) INHALER

(71) Applicant: AstraZeneca AB, Södertälje (SE)

(72) Inventors: Brian FOSTER, Raleigh, NC (US); Dan DEATON, Holly Springs, NC (US); Daniel COONEY, Raleigh, NC (US); James HANNON, Lafayette, CO (US); Eric RICHARDSON, Cave Creek, AZ (US); Paul HAYTON, Bristol (GB); Cal McLENNAN, Chepstow (GB); Pete WILSON, Cambrige (GB); Tom ETHERIDGE, Cambridge (GB); David George ROBINSON, London (GB)

(21) Appl. No.: 18/666,606

(22) Filed: May 16, 2024

(51) Int. Cl. A61M 15/00

(52) U.S. Cl. CPC A61M 15/0031; A61M 15/0036; A61M 2202/064

(57) ABSTRACT

An inhaler includes a main body and a drawer to open out of and close into the main body between an open position and a closed position. The drawer includes a spin chamber having a primary recess to receive air to mix with contents of a capsule and a secondary recess to hold the capsule. When the drawer is in the open position, the secondary recess is exposed to receive a new capsule therein or to withdraw a used capsule therefrom. When the drawer is in the closed position, the capsule is enclosed within the inhaler. The inhaler includes a perforating means to perforate the capsule. The perforating means moves away from a resting position toward a perforating position as the drawer moves into the main body. When set at the perforating position, the perforating means is positioned within the secondary recess to perforate the capsule.

### FIELD OF DISCLOSURE

The present disclosure relates to inhalers, and more specifically, to dry-powder inhalers.

### BACKGROUND

Inhalers are medical devices used to deliver a dose of medicament to a user by inhalation. There are numerous varieties of inhalers, but they all rely on the deliverance of the medicament into a user's lungs where the medicament may then be absorbed. Inhalers are used as a common treatment for asthma and chronic obstructive pulmonary disease (COPD), for example.

Dry powder inhalers are one such variety of inhaler. These deliver medicament to a user in the form of a dry powder, which is advantageous as this allows the medicament to reach further into the lungs than, for instance, metered dose or soft mist inhalers, thereby providing a greater therapeutic benefit to the user.

Existing dry powder inhalers, such as those described in EP 1,270,034 A2 and US 2007/295332 A1, require a user to open the device, insert a capsule, close the device and then manually press at least one button in order to perforate the capsule. Failure to press the buttons with enough force, or failure to press the buttons at all, will result in either a complete failure to perforate the capsule, or a failure to perforate the capsule enough, which may limit the amount of medication that is released. Users must themselves determine the force with which they must press the buttons, which increases the risk of a failed perforation attempt. Additionally, failure to release the buttons may result in a capsule being unable to spin freely, increasing the risk of a failed delivery.

Such inhalers also confine the capsule to a tight space within which it may vibrate or shake in order to release its contents. This makes it less likely that inhalation by a user will successfully remove all the contents of a capsule from the capsule, reducing the overall efficiency of the process.

The present disclosure aims to solve these problems, among others.

### BRIEF SUMMARY

Aspects of the disclosure are as set out in the independent claims and additional features are set out in the dependent claims. Aspects of the disclosure may be provided in conjunction with each other and features of one aspect may be applied to other aspects.

An aspect of the disclosure provides an inhaler comprising: a main body; and a drawer configured to open out of and close into the main body between an open position and a closed position, the drawer comprising: a spin chamber comprising a primary recess for allowing air to mix with contents of a capsule and a secondary recess for holding therein the capsule, wherein when the drawer is in the open position, the secondary recess can be accessed to insert therein a new capsule or to withdraw therefrom a used capsule, and when the drawer is in the closed position, the capsule is enclosed within the inhaler; and perforating means for perforating the capsule, the perforating means arranged such that as the drawer moves into the main body from the open position towards the closed position, the movement of the drawer towards the closed position causes the perforating means to move away from a resting position towards a perforating position, the perforating position being a position within the secondary recess where the perforating means may perforate the capsule, and further movement of the drawer into the main body from the open position towards the closed position causes the perforating means to move from the perforating position to the resting position, such that when the drawer is in the closed position, the perforating means are in the resting position.

Additionally, the perforating means may be configured to interact with a portion of the main body as the drawer moves into the main body from the open position towards the closed position, the interaction causing the perforating means to slide along the portion of the main body.

Additionally, the perforating means may comprise at least one needle configured to transversely slide against the biasing of at least one spring, wherein the at least one spring is in a rest state when the perforating means are in the resting position. A sharp, perforating end of the least one needle may then perforate the capsule. The use of a spring helps to keep the other components of the perforating means in their required positions during the opening and closing of the drawer.

Additionally, the at least one spring may be coupled to a cam post, the cam post adapted to, as the drawer moves into the main body from the open position towards the closed position, slide along the portion of the main body and simultaneously compress the at least one spring, wherein the cam post is coupled to a non-perforating end of the needle.

Additionally, the cam post may comprise a rounded surface, the rounded surface adapted to, as the drawer moves into the main body from the open position towards the closed position, slide along the portion of the main body. The use of a rounded surface may help to minimise the risk of the cam post jamming against the portion of the main body and being unable to move as intended.

Additionally, the cam post may comprise a scraping edge for removing residue from the portion of the main body. This may help to prevent a build-up of residue and helps to ensure that any disruption to air flow through the inhaler is minimised.

Additionally, the scraping edge may be substantially V-shaped. This may help to increase the area over which the scraping edge can remove residue.

Additionally, the portion of the main body may comprise a wedge connected to a flexible arm, and the cam post may slide along a side of the wedge as the drawer moves into the main body from the open position towards the closed position. This may enable the perforating means to perform their function while ensuring that any movement between components does not result in any cracking or breaking.

Additionally, the wedge may comprise a scraping edge for removing residue from the cam post. This may help to prevent a build-up of residue and helps to ensure that any disruption to air flow through the inhaler is minimised.

Additionally, when the drawer is almost in the closed position, the cam post may travel over an end of the wedge, thus causing the at least one spring to decompress. This ensures that the perforating means are moved away from the capsule and returned to their resting position, so that they are in the correct position for a subsequent opening of the drawer.

Additionally, the inhaler may have a longitudinal axis extending from a top of the inhaler, through the main body and the drawer, to a bottom of the inhaler, and as the drawer moves out of the main body from the closed position towards the open position: the cam post may travel over a top surface of the wedge and causes the flexible arm to move downwards from a rest position along the longitudinal axis towards the bottom of the inhaler; and the at least one spring may remain in the rest state. This may enable the drawer to be opened in a controlled manner without any risk of cracking or breaking as the components of the inhaler move.

Additionally, when the drawer is in the open position, the flexible arm may return to the rest position. This ensures that the flexible arm, and the wedge to which it is attached, are both in the correct position for a subsequent closing of the drawer.

Additionally, the at least one needle may comprise a pair of opposing needles, each needle coupled to a respective at least one spring. This may result in two perforations of the capsule, which decreases the time required for the contents of the capsule to be removed from the capsule through inhalation.

Additionally, the pair of opposing needles may be configured to perforate the capsule at the same time. This helps to ensure an efficient and timely emptying of the capsule.

Additionally, the pair of opposing needles may be configured to perforate opposing ends of the capsule. This helps to ensure an efficient and timely emptying of the capsule.

Additionally, the drawer may be coupled to the main body by a hinge mechanism. This enables the drawer to be accessed without having to remove it from the main body entirely.

Additionally, at least a portion of the main body may comprise wax-lubricated PBT.

Additionally, the inhaler may further comprise at least one air inlet adapted to allow air to flow through the inhaler and spin the capsule. This helps to ensure that the inhalation process is as efficient as possible.

### BRIEF DESCRIPTION OF THE FIGURES

Embodiments of the disclosure will now be described, by way of example only, with reference to the accompanying drawings.

FIG. 1A shows a perspective view of an inhaler in accordance with the present disclosure.

FIG. 1B shows a perspective view of an inhaler with an open drawer in accordance with the present disclosure.

FIG. 2 shows an exploded view of an inhaler in accordance with the present disclosure.

FIG. 3A shows a top view of an inhaler with an open drawer in accordance with the present disclosure.

FIG. 3B shows a cross-sectional side view of an inhaler in accordance with the present disclosure.

FIG. 4 shows a cross-sectional view of a spin chamber and perforating means in accordance with the present disclosure.

FIG. 5A is a cross-sectional top view of an inhaler showing the relative positions of the perforating means and the main body of the inhaler when the drawer is in an open position.

FIG. 5B is a cross-sectional top view of an inhaler showing the interaction of the perforating means with the main body of the inhaler as the drawer is being closed into the main body.

FIG. 5C is a cross-sectional top view of an inhaler showing the relative positions of the perforating means and the main body of the inhaler when the drawer is in a closed position.

FIGS. 6A-C are cross-sectional side views showing the interaction of the cam post with the wedge as the drawer is being closed into the main body.

FIG. 7A shows a perspective view of a cam post interacting with the wedge in accordance with the present disclosure.

FIG. 7B shows a perspective view of another type of cam post interacting with the wedge in accordance with the present disclosure.

FIG. 7C shows another perspective view of the cam post of FIG. 7A interacting with the wedge in accordance with the present disclosure.

FIG. 7D shows another perspective view of the cam post of FIG. 7B interacting with the wedge in accordance with the present disclosure.

FIG. 8 shows a cross-sectional side view of the chimney and the drawer being held together in accordance with the present disclosure.

FIGS. 9A-C are a series of cross-sectional side views showing the interaction of the cam post with the wedge as the drawer is moved from a closed position to an open position.

FIG. 10A is a cross-sectional bottom view of an inhaler with an alternative, rotatable perforating means and shows the relative positions of the rotatable perforating means and the main body of the inhaler when the drawer is in an open position.

FIG. 10B is a cross-sectional bottom view of the inhaler of FIG. 10A showing the interaction of the rotatable perforating means with the main body of the inhaler as the drawer is being closed into the main body.

FIG. 10C is a cross-sectional bottom view of the inhaler of FIGS. 10A-B showing the relative positions of the rotatable perforating means and the main body of the inhaler when the drawer is in a closed position.

### DETAILED DESCRIPTION

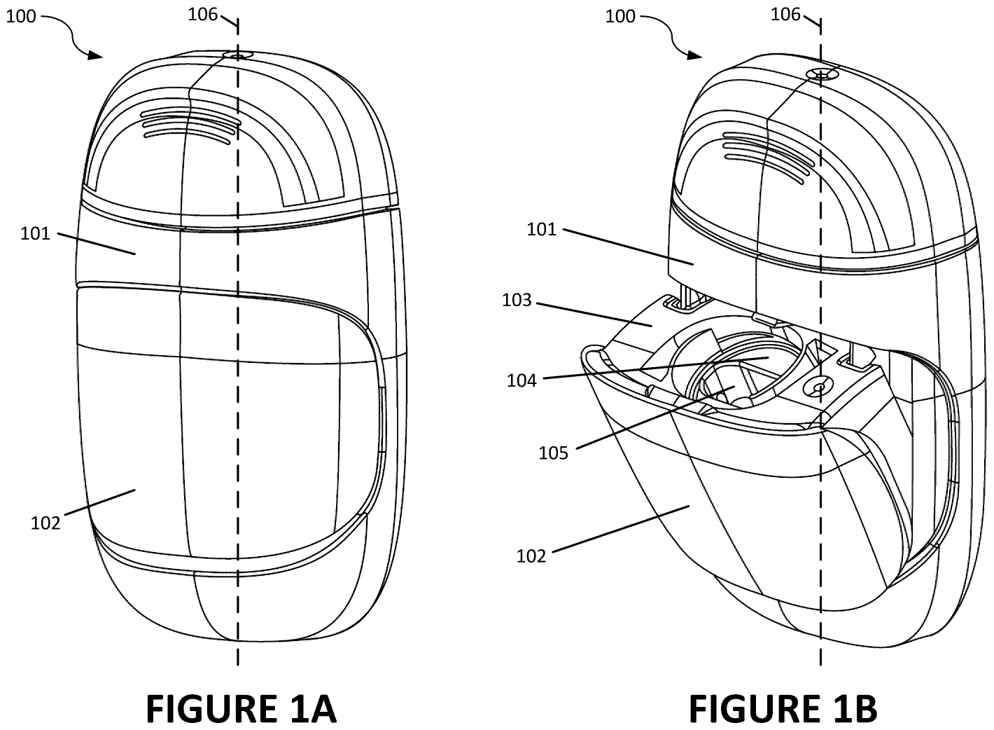

FIGS. 1A-B show perspective views of an inhaler 100. The inhaler 100 comprises a main body 101 and a drawer 102. The drawer 102 may be coupled to the main body by way of a hinge mechanism, which allows the drawer 102 to open out of and close into the main body 101 of the inhaler 100. This enables the drawer 102 to be accessed without having to remove it from the main body 101 entirely. The inhaler 100 may comprise a longitudinal axis 106, with the top of the inhaler 100 being positioned above the bottom of the inhaler 100 with respect to the longitudinal axis 106.

In FIG. 1A, the drawer 102 is shown as being in a closed position. In the closed position, a longitudinal axis of the drawer 102 may directly correspond to the longitudinal axis 106 of the inhaler 100. In FIG. 1B, the drawer 102 is shown as being in an open position, such that the components of the drawer 102 are visible. In the open position, the drawer 102 is angled outwards such that the longitudinal axis of the drawer is angled away from the longitudinal axis 106 of the inhaler 100.

The main body 101 is configured to act as a framework for the inhaler 100 and enclose the majority of the other components of the inhaler 100. The main body 101 may comprise polybutylene terephthalate (PBT) and at least a portion of the main body 101 may comprise wax-lubricated PBT. The main body 101 may comprise at least one air inlet to allow air to flow through the inhaler 100. The drawer 102 is configured to be opened out of and closed into the main body 101. More specifically, the spin chamber 103 of the drawer 102 is configured to receive a capsule and to allow the contents of the capsule to mix with air during inhalation. The contents of the capsule may comprise medicament in the form of a dry powder.

The drawer 102 comprises a spin chamber 103, which is located near the top of the drawer 102 with respect to the longitudinal axis 106. The spin chamber may comprise a primary recess 104 and a secondary recess 105. The primary recess 104 may extend downwards from the top surface of the spin chamber 103. The secondary recess 105 may be located within a bottom surface of the primary recess 104. As such, the secondary recess 105 can be considered as an extension of the primary recess 104. The primary recess 104 may be substantially cylindrical in shape and the secondary recess 105 may be substantially obround in shape. The primary recess 104 has a larger volume than the secondary recess 105.

The secondary recess 105 is configured to receive the capsule. The primary recess 104 is configured to allow the contents of the capsule to mix with air during inhalation.

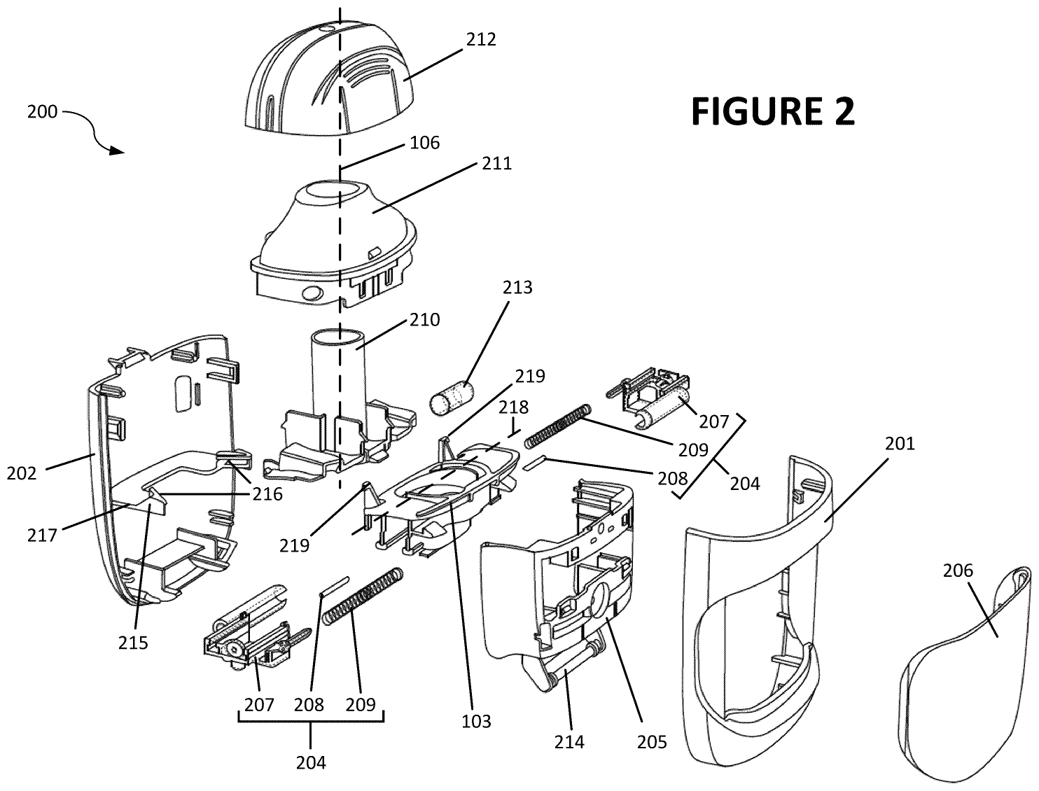

Use of the inhaler 100 begins with the insertion of a capsule into the drawer 102. The capsule is placed into the secondary recess 105 and the drawer 102 is closed into the main body 101. Closing the drawer 102 causes the capsule to be perforated, which will be described in greater detail with respect to FIG. 2. As the user inhales through the mouthpiece (not shown), the air flow through the spin chamber 103 causes the capsule to be lifted out of the secondary recess 105 into the primary recess 104, where it may spin such that its contents may mix with air flowing through the spin chamber 103. This mixture is then inhaled by the user. The drawer 102 may then be opened and the capsule removed.

The inhaler 100 and its use will be described in greater detail with respect to FIG. 2.

FIG. 2 shows an exploded view of an inhaler 200. The inhaler 200 may correspond to the inhaler 100 from FIGS. 1A-B and may therefore comprise a main body and a drawer, corresponding respectively to the main body 101 and the drawer 102 from FIGS. 1A-B.

More specifically, the main body of inhaler 200 may comprise a front casing 201 and a rear casing 202. The front casing 201 and rear casing 202 are connected to each other to provide a space within which other components of the inhaler 200 may be located. Each of the front casing 201 and rear casing 202 comprises an inner surface and an outer surface. When the front casing 201 and rear casing 202 are connected to each other, the two inner surfaces face inwards towards each other, while both outer surfaces face outwards. The front casing 201 and rear casing 202 both extend upwards along a longitudinal axis that may correspond to the longitudinal axis 106 from FIGS. 1A-B.

The front casing 201 comprises an aperture through which the drawer may move between an open position and a closed position. When the drawer is in the closed position, an outer surface of the drawer casing substantially fills the aperture of the front casing 201. When the drawer is in the open position, the top surface of the spin chamber is exposed, such that a capsule 213 may be inserted into or removed from the drawer. As described, the capsule 213 may contain medicament in the form of a dry powder. When the drawer is in the open position, the side walls of the supporting framework 205 may substantially block access to other internal components of the drawer.

The rear casing 202 may comprise at least one wedge 215, the at least one wedge 215 comprising an inner side 216 and being connected to a flexible arm 217. FIG. 2 shows an embodiment in which the rear casing 202 comprises two wedges 215, each comprising an inner side 216 and each attached to a separate flexible arm 217, but it is to be understood that fewer or more wedges 215 and flexible arms 217 are possible. In FIG. 2, the flexible arms 217 protrude outwards from the inner surface of the rear casing 202 along an axis that is substantially perpendicular to the longitudinal axis 106 of the rear casing 202.

The drawer of inhaler 200 may comprise a spin chamber 103, perforating means 204, a supporting framework 205 and a drawer casing 206. The spin chamber 103 may correspond to the spin chamber 103 from FIG. 1B and may comprise a transverse axis 218 that is substantially perpendicular to the longitudinal axis 106 of the inhaler and also substantially perpendicular to the axis along which the flexible arm 217 protrudes. The spin chamber 103, in addition to comprising a primary recess 104 and a secondary recess 105 for receiving a capsule 213, may also comprise at least one guide post 219. FIG. 2 shows an embodiment in which the spin chamber 103 comprises two guide posts 219, each located on opposing sides of the spin chamber 103 along the transverse axis 218. The guide posts 219 may extend upwards from a top surface of the spin chamber 103 substantially along the longitudinal axis 106.

The spin chamber 103 is coupled to the perforating means 204, which are positioned at a side of the spin chamber 103 along the transverse axis 218. The perforating means 204 are positioned so as to be able to move along the transverse axis 218 between a resting position and a perforating position. The perforating position is a position within the secondary recess 105 where the perforating means 204 may perforate the capsule 213. When in the resting position, the perforating means are further away from the center of the spin chamber 103 than when in the perforating position. The spin chamber 103 may comprise rails to allow the perforating means 204 to slide along the transverse axis 218 between the resting position and the perforating position. The perforating means 204 may comprise grooves that interact with the rails of the spin chamber 103 to enable this movement. The spin chamber 103 may also comprise a T-rail (not shown) that helps to maintain alignment of the spin chamber 103 and the perforating means 204. The spin chamber 103 may further comprise perforating means retention clips (not shown) that prevent the perforating means 204 from moving outwards beyond their resting position along the transverse axis 218.

The spin chamber 103 and perforating means 204 may be coupled to the supporting framework 205, which holds the spin chamber 103 in a set position within the drawer. The supporting framework 205 also encloses the perforating means 204 within the drawer and may also help to prevent the perforating means 204 from moving outwards beyond their resting position along the transverse axis 218. A front side of the supporting framework 205 is attached to the drawer casing 206. The supporting framework may also comprise a hinge 214, which may be connected to the front casing 201 by way of a hook mechanism. The hook mechanism may have a substantially semi-circular cross section. The hinge 214 may also be connected to the rear casing 202. The presence of the hinge 214 may enable the drawer to be opened out of and closed into the main body while remaining attached to the main body. This enables the drawer to be accessed without having to remove it from the main body entirely.

The perforating means 204 may comprise a cam post 207, a needle 208 and a spring 209. The cam post 207 is coupled to a non-perforating end of the needle 208 and to a first end of the spring 209. The needle 208 and spring 209 both extend away from the cam post 207 along the transverse axis 218. The needle 208 may be encompassed by the spring 209, or it may be positioned away from the spring 209.

The second end of the spring 209 may be coupled to an inner portion of the perforating means 204, whereas the perforating end of the needle 208 is not directly connected to any other part of the inhaler. The spring 209 is in a rest state when the drawer is in the open position and when the drawer is in the closed position, but may be compressed as the drawer moves from the open position to the closed position, as will be described in greater detail.

FIG. 2 shows the perforating means 204 as comprising two sets of cam posts 207, needles 208 and springs 209, with each set located along the transverse axis 218 on opposing sides of the spin chamber 103, although the preceding paragraphs have so far described only one cam post 207, one needle 208 and one spring 209. It is to be understood that the inhaler may function with one cam post 207, one needle 208 and one spring 209, or with two cam posts 207, two needles 208 and two springs 209. The only requirements are that the perforating means comprises at least one cam post 207, at least one needle 208 and at least one spring 209. In an embodiment, the perforating means comprises two cam posts 207, two needles 208 and two springs 209, as shown in FIG. 2. In this embodiment, each of the two springs 209 may be coupled to the same inner portion of the perforating means at their respective second ends.

The two needles 208 may comprise a pair of opposing needles 208, each needle 208 coupled to a respective spring 209. The use of two opposing needles 208 may result in two perforations of the capsule 213. This decreases the time required for the contents of the capsule 213 to be removed from the capsule 213 through inhalation, since there will be two holes created in the capsule 213. The opposing needles 208 may be configured to perforate the capsule 213 at the same time. This helps to ensure an efficient and timely emptying of the capsule 213, since both holes will be created at the same time.

As described, the secondary recess 105 may be substantially obround-shaped. The needles 208 may be configured to enter opposing ends of the secondary recess 105 and subsequently perforate opposing ends of the capsule 213. This helps to ensure an efficient and timely emptying of the capsule, since this minimizes the distance the contents of the capsule 213 will have to travel in order to exit the capsule 213.

As described, the perforating means 204 are configured to move along the transverse axis 218 between a resting position and a perforating position. More specifically, the cam post may be configured to transversely slide against the bias of the spring 209, which causes the spring 209 to compress. Since the needle 208 is attached to the cam post 207, the needle 208 may also be configured to transversely slide against the bias of the spring 209.

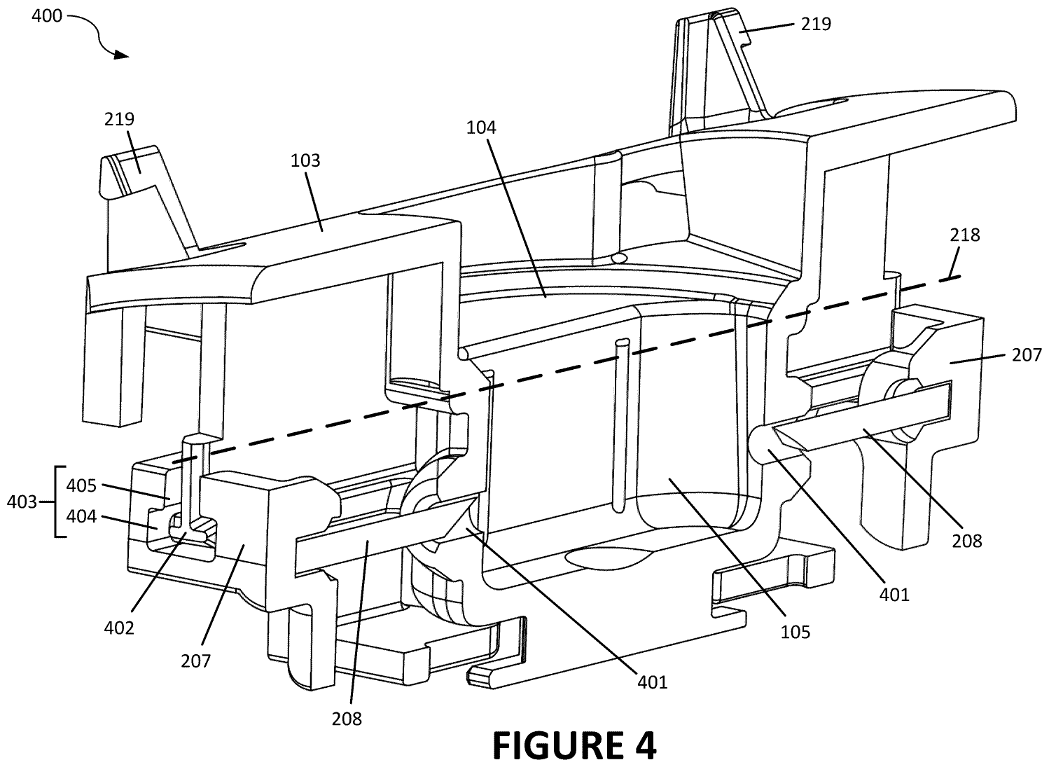

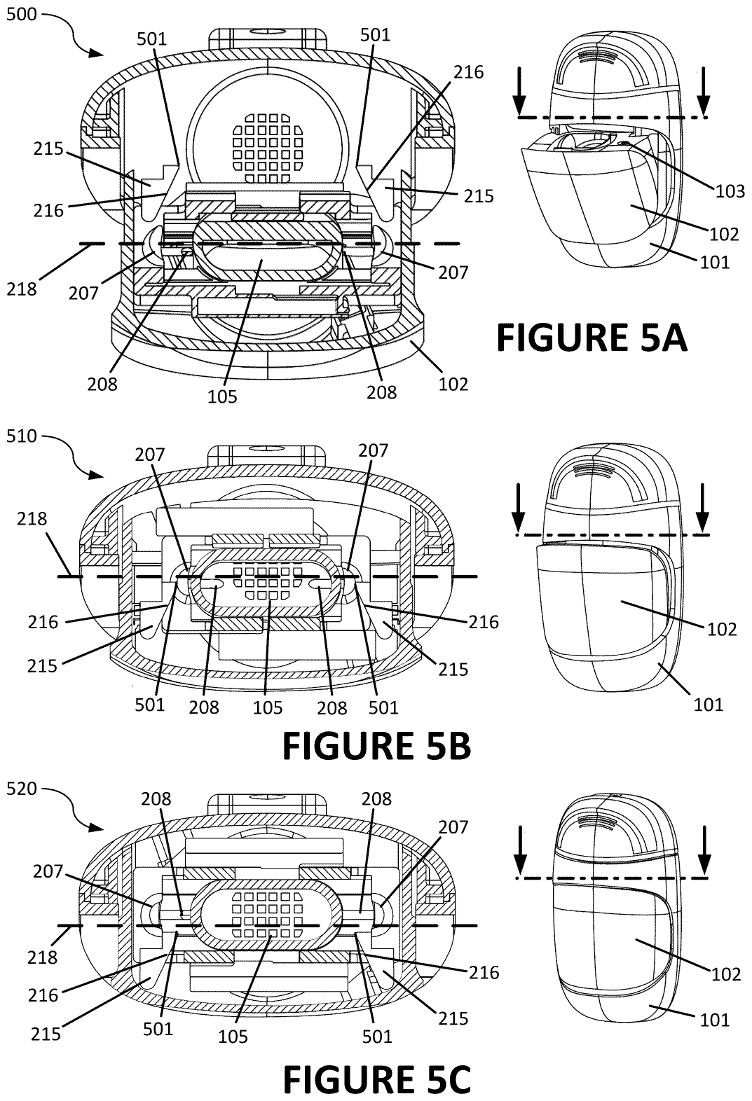

As will be described in greater detail with respect to FIGS. 4 and 5A-C, the movement of the drawer from an open position to a closed position may cause the perforating means 204 to move from the resting position to the perforating position.

The inhaler 200 may further comprise an inhalation chimney 210. The inhalation chimney 210 may comprise a hollow tube through which air and medicament may pass. The inhalation chimney 210 is positioned along the longitudinal axis 106 near the top of the inhaler, such that when the drawer is in the closed position, the inhalation chimney is directly above the spin chamber 103. The hollow tube extends along the longitudinal axis 106. The bottom of the hollow tube of the chimney 210 aligns with the primary recess 104 and secondary recess 105 of the spin chamber 103. When the drawer is in the closed position, the inhalation chimney 210 and the spin chamber 103 together define a space within which the contents of the capsule 213 may be spun as air travels through the inhaler 200.

The inhalation chimney 210 may also comprise at least one protruding rib along which the at least one guide post 219 of the spin chamber 103 may pass. The at least one protruding rib may extend outwards along the transverse axis 218. For example, there may be two protruding ribs on opposing sides of the inhalation chimney 210. The number of protruding ribs is the same as the number of guide posts 219.

The inhalation chimney 210 may also comprise at least one drawer retention clip (not shown). The at least one drawer retention clip may be situated near the bottom of the inhalation chimney 210 on the side that is closest to the rear casing 202. In an embodiment, the at least one drawer retention clip comprises two drawer retention clips on opposing sides of the inhalation chimney 210 with respect to the transverse axis 218.

The inhaler 200 may also comprise a mouthpiece 211. The mouthpiece 211 is positioned on top of the inhalation chimney 210 and comprises an aperture through which the inhalation chimney 210 may extend. The inhalation chimney 210 may move upwards along the longitudinal axis 106 such that a top surface of the inhalation chimney 210 is higher than a top surface of the mouthpiece 211 with respect to the longitudinal axis 106. The inhalation chimney 210 may move downwards along the longitudinal axis 106 such that the top surface of the inhalation chimney 210 is at the same level as the top surface of the mouthpiece 211 with respect to the longitudinal axis 106.

The mouthpiece 211 is attached to the front casing 201 and rear casing 202 of the inhaler 200.

The inhaler 200 may also comprise a cap 212. The cap 212 is positioned on top of the mouthpiece 211 and may cover the entire top surface of the mouthpiece 211. The cap 212 is attached to the mouthpiece 211 by way of a hinge mechanism that enables the cap 212 to either allow access to the mouthpiece 211 or to cover and prevent access to the mouthpiece 211.

With reference now to the function of the components of the inhaler 200, the front casing 201 and rear casing 202 are configured to act as the main body of the inhaler 200. The front casing 201 and rear casing 202 are joined to define an outer housing of the inhaler 200, within which other components may be enclosed.

The spin chamber 103, as has been described with reference to FIG. 1B, is configured to receive a capsule 213 and to allow air to mix with the contents of the capsule 213. More specifically, the secondary recess 105 of the spin chamber 103 is configured to receive the capsule 213. As air flows through the inhaler 200, the capsule 213 may be lifted out of the secondary recess 105 and into the primary recess 104, where the capsule 213 may spin around in order to allow its contents to mix with the air.

The perforating means 204 are configured to perforate the capsule 213, thus releasing the contents of the capsule 213 and allowing them to mix with air so that they may be inhaled by a user. More specifically, the perforating means 204 are configured to move inwards along the transverse axis 218 from a resting position to a perforating position as the drawer moves from an open position to a closed position. When at the perforating position, which occurs shortly before the drawer is in the closed position, the perforating means 204 are configured to perforate the capsule 213 and then move back from the perforating position to the resting position. When the drawer is in the closed position, the perforating means 204 are in the resting position. As the drawer moves from the closed position to the open position, the perforating means are configured to remain in the resting position.

The perforating means 204 are configured to interact with a portion of the main body of the inhaler 200 as the drawer moves between the open position and the closed position, which causes the perforating means to move away from their resting position towards their perforating position. More specifically, the perforating means are configured to interact with the wedge 215, which is attached to the flexible arm 217.

As the drawer moves into the main body of the inhaler 200 from the open position to the closed position, the cam post 207 of the perforating means 204 is configured to slide along the inner side 216 of the wedge 215. The angle of this inner side 216 causes the cam post 207 to be pushed inwards towards the center of the spin chamber 103 along the transverse axis 218, against the biasing of the spring 209. This compresses the spring 209, which subsequently provides a resistive force. This helps to keep the other components of the perforating means 204 in the desired position. The needle 208, which is attached to the cam post 207, also moves inwards towards the center of the spin chamber 103 and passes through a small aperture in the side of the spin chamber 103. Further details of this small aperture will be discussed with reference to FIG. 4. By the time the cam post 207 has reached the end of the inner side 216, the perforating means 204 have moved along the transverse axis 218 and have reached their perforating position. When in the perforating position, the needle 208 has extended through the small aperture in the side of the spin chamber 103 and into the secondary recess 105, where it may perforate the capsule 213. This process is described in greater detail with respect to FIG. 4 and FIGS. 5A-C.

This means that a capsule 213 can be perforated as the drawer is closed into the main body, rather than this being a separate step that must be initiated after the drawer has been closed. This makes use of the inhaler 200 easier and quicker for a user and also minimises the risk of a user failing to perforate a capsule (e.g. by not pressing a button hard enough), since the perforating means 204 must reach the perforating position in order for the drawer to successfully close.

Once the perforating position has been reached and the capsule 213 has been perforated, the perforating means 204 are configured to pass over the edge of the inner side 216 of the wedge 215 and in doing so return to the resting position. The compressed spring 209 decompresses and returns to its rest position. In doing so, the spring 209 pushes the needle 208 out of the secondary recess 105 such that the perforating means 204 can return to the resting position so that they are in the correct position for a subsequent opening of the drawer. At this point, the drawer is in the closed position. Beneficially, this means that a user does not have to manually reset the perforating means 204.

As the drawer is moved from a closed position to an open position, the perforating means 204 are configured to interact with the wedge 215, but in a different manner to the interaction that takes place when the drawer is being closed. As the drawer moves away from the closed position, the perforating means 204 are configured to travel over a top surface of the wedge 215. More specifically, the cam post 207 travels over the top surface of the wedge 215, which causes the flexible arm 217 to move downwards along the longitudinal axis 106 towards the bottom of the inhaler. As the cam post 207 travels over the wedge 215, the perforating means 204 remain in the resting position with respect to the transverse axis 218, meaning that the spring remains in the rest state. Once the cam post 207 has travelled over the top surface of the wedge 215, the wedge 215 moves back up to its normal resting position so that it is in the correct position for a subsequent closing of the drawer.

The inhalation chimney 210 is configured to move downwards with respect to the longitudinal axis 106 as the drawer moves from an open position to a closed position and is configured to move upwards with respect to the longitudinal axis 106 as the drawer moves from a closed position to an open position. More specifically, the guide posts 219 of the spin chamber are configured to interact with the inhalation chimney 210 as the drawer moves between the open and closed positions, which causes the inhalation chimney 210 to move upwards or downwards. When the drawer is in the closed position, the drawer retention clips are configured to hold the guide posts 219 in position, such that a force is required to move the guide posts 219 out of this position and open the drawer.

The mouthpiece 211 is configured to be inserted into a user's mouth during inhalation. The cap 212 is configured to cover the mouthpiece 211 when the inhaler 200 is not in use, thus preventing any foreign substances from entering the inhaler 200 through the mouthpiece 211.

In order to use the inhaler 200, a user may insert a capsule 213 into the secondary recess 105 of the spin chamber 103. The drawer must be in the open position for this to take place, since the spin chamber 103 cannot be accessed if the drawer is in the closed position. Once the capsule 213 is positioned within the secondary recess 105, the user may push the drawer inwards to move it from the open position towards the closed position. As the drawer moves towards the closed position, the perforating means 204 interact with the wedge 215, which causes them to slide along the inner side 216 of the wedge 215 and to move inwards along the transverse axis 218, as has been described and as will be described in greater detail with respect to FIGS. 4 and 5A-C.

The movement of the drawer causes the cam post 207 and the needle 208 to move inwards towards the center of the spin chamber 103 along the transverse axis 218. As the cam post 207 approaches the edge of the wedge 215, the needle 208 perforates the capsule 213. The perforating means 204 then pass over the edge of the wedge 215 and return to the resting position.

The spin chamber 103 also interacts with the inhalation chimney 210 as the drawer moves from the open position towards the closed position. More specifically, the guide posts 219 of the spin chamber 103 slide along the protruding ribs of the inhalation chimney before travelling over sealing ramps of the inhalation chimney 210 as the drawer approaches the closed position. As the guide posts 219 travel over the sealing ramps, they cause the inhalation chimney 210 to be pulled downwards along the longitudinal axis 106, such that a bottom surface of the inhalation chimney 210 is brought closer to a top surface of the spin chamber 103. The two surfaces may be brought into contact, or a small gap may remain between them when the drawer is in the closed position. When the drawer is in the closed position, the inhalation chimney 210 has been pulled down such that a top surface of the inhalation chimney 210 is level with a top surface of the mouthpiece 211 with respect to the longitudinal axis 106. Drawer retention clips hold the guide posts 219 in position, such that the inhalation chimney 210 is held in position with respect to the spin chamber 103.

As discussed above, when the drawer is in the closed position, the perforating means 204 have perforated the capsule 213 and returned to the resting position and the inhalation chimney 210 has moved down towards the spin chamber 103. At this stage, the user may open the cap 212 to expose the mouthpiece 211. By placing the inhaler 200 in their mouth, tilting it and inhaling, an air flow may be generated through the inhaler 200. The air flow may lift the perforated capsule 213 out of the secondary recess 105 and into the primary recess 104 of the spin chamber 103, where it may cause the capsule 213 to spin and the contents of the capsule 213 to mix with the air. The resulting mixture of the contents of the capsule 213 and the air may then pass through the hollow tube of the inhalation chimney 210, through the aperture of the mouthpiece 211 and into the mouth of the user.

Upon successful inhalation, the drawer of the inhaler 200 may then be opened so that the capsule 213 may be removed. As the drawer is pulled outwards, the guide posts 219 push the drawer retention clips away and travel back over the sealing ramps. The guide posts 219 then interact with the protruding ribs of the inhalation chimney 210, which pushes the inhalation chimney 210 upwards with respect to the longitudinal axis 106.

At the same time, the perforating means 204 travel over the wedges 215. This movement pushes the wedges 215 downwards with respect to the longitudinal axis 106. The perforating means 204 therefore remain in the resting position as they travel over the wedges 215.

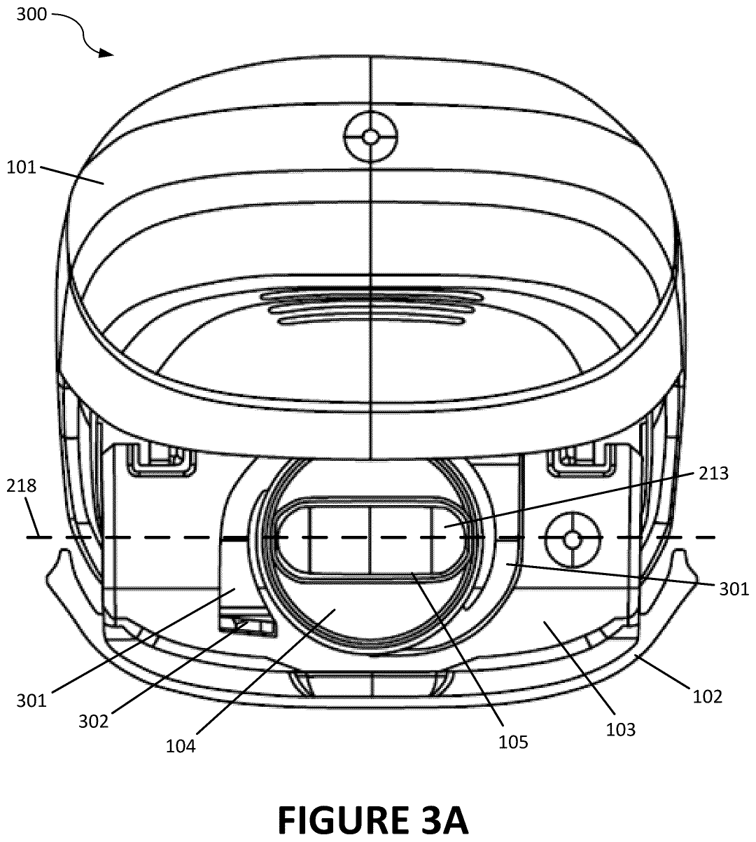

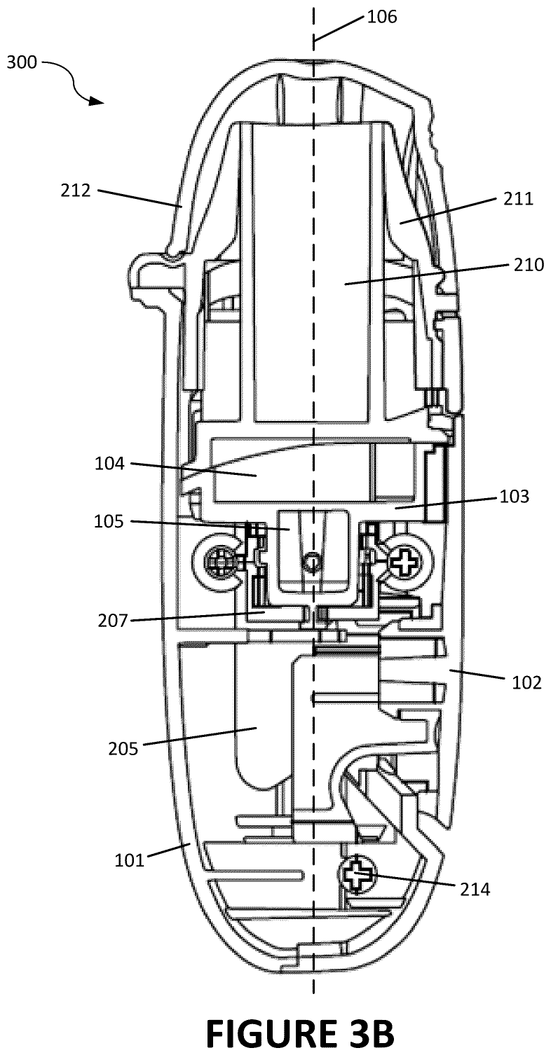

FIG. 3A shows a top view of an inhaler 300 with an open drawer in accordance with the present disclosure. FIG. 3B shows a cross-sectional side view of an inhaler 300 in accordance with the present disclosure. The inhaler 300 may be the same as the inhaler 100 from FIG. 1 and the inhaler 200 from FIG. 2.

Referring firstly to FIG. 3A, the spin chamber 103 is shown in greater detail. As has been discussed, the spin chamber 103 comprises a primary recess 104 and a secondary recess 105. The secondary recess 105 is configured to receive the capsule 213.

The spin chamber 103 may also comprise at least one curved channel 301, through which air may travel from at least one air inlet 302 into the primary recess 104. The air may then mix with the contents of the capsule 213 during inhalation. FIG. 3A shows an embodiment in which the spin chamber 103 comprises two curved channels 301. The curved channels 301 may be separated from the primary recess 104 along a majority of their length by a curved wall.

Referring now to FIG. 3B, the internal structure of the inhaler 300 when in the closed position is shown. As has been described with reference to FIG. 2, the components of the drawer 102 are enclosed within the main body 101 when the drawer 102 is in the closed position. The inhalation chimney 210 may extend through the mouthpiece 211, which itself is covered by the cap 212. The spin chamber 103 is positioned at the top of the drawer 102, such that when the drawer 102 is in the closed position, the spin chamber 103 is directly underneath the inhalation chimney 210 with respect to the longitudinal axis 106. The spin chamber 103 is coupled to the supporting framework 205, which is attached to the main body 101 by way of a hinge mechanism 214. The cam post 207 is coupled to the side of the spin chamber 103 and is able to move inwards with respect to the transverse axis, but not upwards or downwards with respect to the longitudinal axis 106.

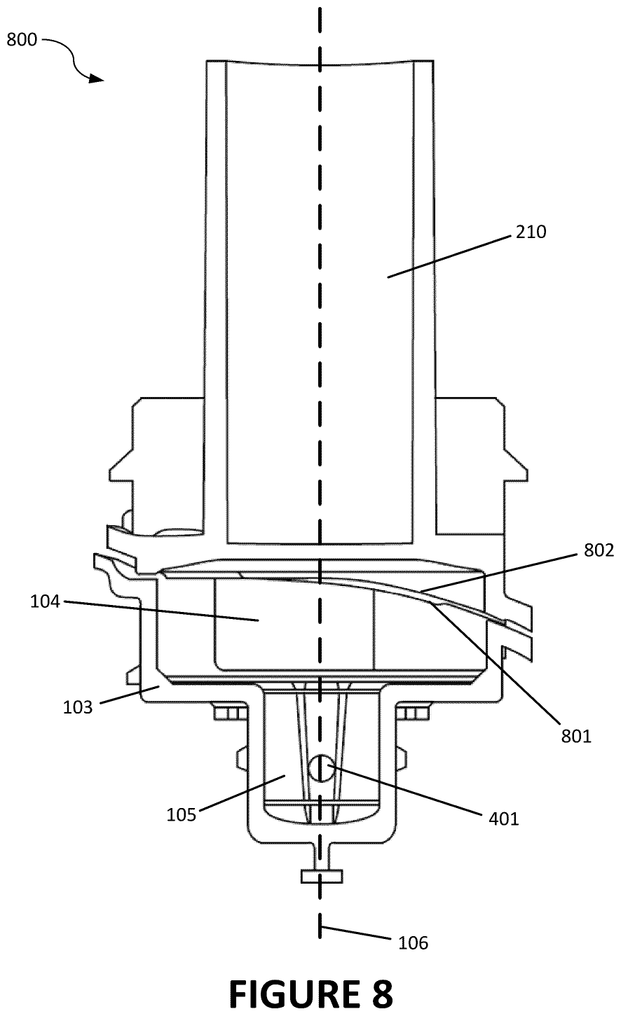

Referring now to both FIGS. 3A and 3B, as has been described and will be described in greater detail with respect to later Figures, when the drawer 102 is closed into the main body 101 from an open position towards a closed position, the perforating means are configured to interact with a portion of the main body 101, which causes the perforating means to move from a resting position to a perforating position, where the perforating means may perforate a capsule held in the secondary recess 105. As the drawer 102 continues to move towards the closed position, the perforating means return to the resting position and the inhalation chimney 210 is pulled downwards with respect to the longitudinal axis 106, such that a chamber may be defined. As will be described with reference to FIG. 8, this chamber may comprise the primary recess 104, the secondary recess 105 and a volume defined by the inhalation chimney 210. During inhalation, air may travel through the air inlets 302, along the curved channels 301 and into the primary recess 104, into which the capsule 213 has been lifted and the contents of the capsule have begun to empty. The air may then mix with the contents of the capsule 213 as the capsule 213 is spun around by the air.

FIG. 4 shows a cross-sectional view 400 of the spin chamber 103 and part of the perforating means. The perforating means may be the same as the perforating means 204 from FIG. 2.

As has been described, the spin chamber 103 comprises a primary recess 104 and a secondary recess 105. The primary recess 104 may have a curved wall and be substantially cylindrical in shape. The secondary recess 105 is smaller than the primary recess 104 and may be located within a bottom surface of the primary recess 104—in this way, the secondary recess 105 can be considered as an extension of the primary recess 104. The secondary recess 105 may have a length along the transverse axis 218 and a depth along the longitudinal axis 106 and may be substantially obround-shaped such that the length is greater than the depth.

The spin chamber 103 may also comprise at least one guide post 219-FIG. 4 shows an embodiment in which the spin chamber 103 comprises two guide posts 219. Both guide posts 219 extend upwards from a top surface of the spin chamber 103 along the longitudinal axis 106 and are situated on the same side of the spin chamber 103, but at opposing ends with respect to the transverse axis 218.

The spin chamber 103 may also comprise at least one small aperture 401 in the side of the secondary recess 105. In an embodiment, the spin chamber 103 may comprise two small apertures 401 at opposing ends of the secondary recess 105. The two small apertures 401 may have the same dimensions and be positioned level with one another along the transverse axis 218. The two small apertures 401 may also be positioned at a same depth of the secondary recess 105 with respect to the longitudinal axis 106.

The spin chamber 103 may also comprise rails 402 near the bottom of the spin chamber 103 with respect to the longitudinal axis 106. The rails 402 may extend in a direction parallel to the transverse axis 218, from one end of the spin chamber 103 to the other. In an embodiment, the spin chamber may comprise first and second rails 402 (only one is shown in FIG. 4 due to the figure being a cross-sectional view). The first and second rails 402 may be parallel and may be located at the same depth with respect to the longitudinal axis 106. The first and second rails 402 may also be positioned on opposing sides of the spin chamber 103, such that when the drawer is in the closed position, the first rail 402 is closer to the rear casing than the front casing and the second rail 402 is closer to the front casing than the rear casing. Each of the first and second rails 402 may comprise one interrupted rail, or may comprise a plurality of interrupted rails extending along the same axis. Each of the first and second rails 402 may have inverted T-shape cross-sections.

As has been discussed, the perforating means may comprise two cam posts 207, two needles 208 and two springs (not shown in FIG. 4). Each needle 208 is attached at its non-perforating end to a cam post 207 and each needle 208 is aligned with one of the small apertures 401. When in the resting position, the perforating means may be arranged such that the needle 208 is within the small aperture 401 (but not extending all the way through) or outside the small aperture 401.

Each cam post 207 may further comprise grooves 403 extending down from a top surface of the cam post 207. In an embodiment, each cam post 207 may comprise first and second grooves 403. In this embodiment, the first and second groove 403 may each comprise a lower section 404 with a substantially rectangular cross section and an upper section 405 with a substantially rectangular cross section that is different to the cross section of the lower section 404. The lower section 404 may be wider than the upper section 405, such that the first and second grooves 403 each have inverted T-shape cross-sections which correspond to the inverted T-shape cross-sections of the rails 402 of the spin chamber 103.

The first and second grooves 403 may be positioned on opposing sides of the cam post 207, such that when the drawer is in the closed position, the first groove 403 is closer to the rear casing than the front casing and the second groove 403 is closer to the front casing than the rear casing. The distance between the first and second grooves 403 may be substantially the same as the distance between the first and second rails 402, which enables the first groove 403 to fit around the first rail 402 and the second groove 403 to fit around the second rail 402.

As has already been described, the spin chamber 103 is configured to receive a capsule and to allow air to mix with the contents of the capsule. More specifically, the secondary recess 105 is configured to receive and hold therein the capsule, while the primary recess 104 is configured to allow air to mix with the contents of the capsule when the inhaler is in use. The spin chamber 103 is designed so as to allow a capsule to be lifted out of the secondary recess 105 and spun around in the primary recess 104.

The guide posts 219 of the spin chamber 103 are configured to interact with the inhalation chimney of the inhaler. More specifically, the guide posts 219 are configured to pull the inhalation chimney down with respect to the longitudinal axis 106 as the drawer is moved from an open position to a closed position. The guide posts 219 are also configured to interact with the inhalation chimney to hold the inhalation chimney and the spin chamber 103 when the drawer is in the closed and to push the inhalation chimney upwards with respect to the longitudinal axis 106 as the drawer is moved from the closed position to the open position. This movement of the inhalation chimney means that when a user inhales through the mouthpiece, a top surface of the chimney is level with a top surface of the mouthpiece, thus providing a more comfortable experience.

As has been discussed and will be discussed further with respect to FIGS. 5A-C, the perforating means are configured to interact with the main body of the inhaler and perforate the capsule so that its contents can be mixed with air during inhalation.

The small apertures 401 are configured to allow the needles 208 access to the secondary recess 105 so that the capsule can be perforated.

The first and second rails 402 of the spin chamber are configured to fit into the first and grooves 403 of the cam post 207 respectively so that the cam post 207 can slide along the first and second rails 402 and move between its resting position and its perforating position. The cross-sections of the rails 402 and of the grooves 403 are configured to complement one another such that the cam post 207 can slide along the rails 402 smoothly without wobbling and without the risk of falling off.

When the drawer is moved from an open position to a closed position, the perforating means are configured to interact with a portion of the main body of the inhaler, as has been discussed. The cam post 207 interacts with the wedge (not shown in FIG. 4), which causes the cam post to slide along the first rail 402 of the spin chamber 103 and move inwards towards the center of the spin chamber 103 along the transverse axis 218. As this occurs, the needle 208 is also moved along the transverse axis 218 and passes through the small aperture 401 and into the secondary recess 105, where it may perforate the capsule. Once the capsule has been perforated and the cam post 207 has passed over the edge of the wedge, the needle 208 retracts out of the secondary recess 105 and back through the small aperture 401 to its resting position. This means that when the time comes to remove the capsule 213 and replace it with a new capsule 213, there is no chance of a user coming into contact with the needle 208.

FIG. 5A is a cross-sectional top view 500 of an inhaler showing the relative positions of the perforating means and the main body of the inhaler when the drawer is in an open position.

FIG. 5B is a cross-sectional top view 510 of an inhaler showing the interaction of the perforating means with the main body of the inhaler as the drawer is being closed into the main body.

FIG. 5C is a cross-sectional top view 520 of an inhaler showing the relative positions of the perforating means and the main body of the inhaler when the drawer is in a closed position.

In each of FIGS. 5A-C, the inhaler may be the same as inhalers 100, 200 or 300.

Starting at FIG. 5A, the drawer 102 is shown to be in the open position. When in the open position, the drawer 102 cannot be moved any further out of the main body 101 and the spin chamber 103 is exposed so that a capsule (not shown) can be inserted into the secondary recess 105 of the spin chamber 103. The perforating means 204 are in a resting position and cannot be moved any further away from the center of the spin chamber 103 along the transverse axis 218.

As has already been described with reference to FIG. 2 and FIG. 4, the perforating means 204 comprise at least one cam post 207, at least one needle 208 and at least one spring 209. FIGS. 5A-C show an embodiment in which the perforating means 204 comprise two cam posts 207, two needles 208 and two springs (not shown in FIGS. 5A-C). In the open position, the perforating means 204 are arranged such that the at needles 208 are outside the secondary recess 105 and are unable to perforate the capsule located in the secondary recess 105. Each needle 208 directly opposes the other along the transverse axis 218.

The cam posts 207 are not yet in contact with the wedges 215, which each comprise an inner side 216 and an edge 501.

FIG. 5B shows the drawer 102 as it is moving from an open position towards a closed position. The movement of the drawer 102 causes the perforating means 204 to interact with a portion of the main body of the inhaler. More specifically, the cam posts 207 interact with the wedges 215. Each cam post 207 interacts with its respective wedge 215 at the same time and travels along the inner side 216 of its respective wedge 215.

Due to the shape of the wedge 215, as each cam post 207 slides along the inner side 216 of its respective wedge 215, the perforating means 204 are pushed inwards towards the center of the spin chamber 103 along the transverse axis 218.

Since each cam post 207 is attached to a respective needle 208, each needle 208 also moves inwards along the transverse axis 218 as the drawer 102 moves towards the closed position. The needles 208 pass through the small apertures in the spin chamber and enter the secondary recess 105. At this point, the perforating means 204 are in the perforating position, and may perforate the capsule that is located in the secondary recess 105. Beneficially, this process takes place as the drawer 102 is being closed into the main body 101, without the need for the user to press a button or instigate perforation by any other means. In FIG. 5B, the perforating means 204 are in the perforating position.

FIG. 5C shows the perforating means 204 after perforation, when the drawer 102 is in the closed position. The continued movement of the drawer 102 towards the closed position causes the cam posts 207 to pass over the edges 501 of their respective wedges 215. Once the cam posts 207 have passed over these edges 501, the springs 209, which were compressed during perforation, may decompress and cause the perforating means 204 to return to the resting position with respect to the transverse axis 218, so that they are in the correct position for a subsequent opening of the drawer. The cam posts 207 are then held in place by the wedges 215.

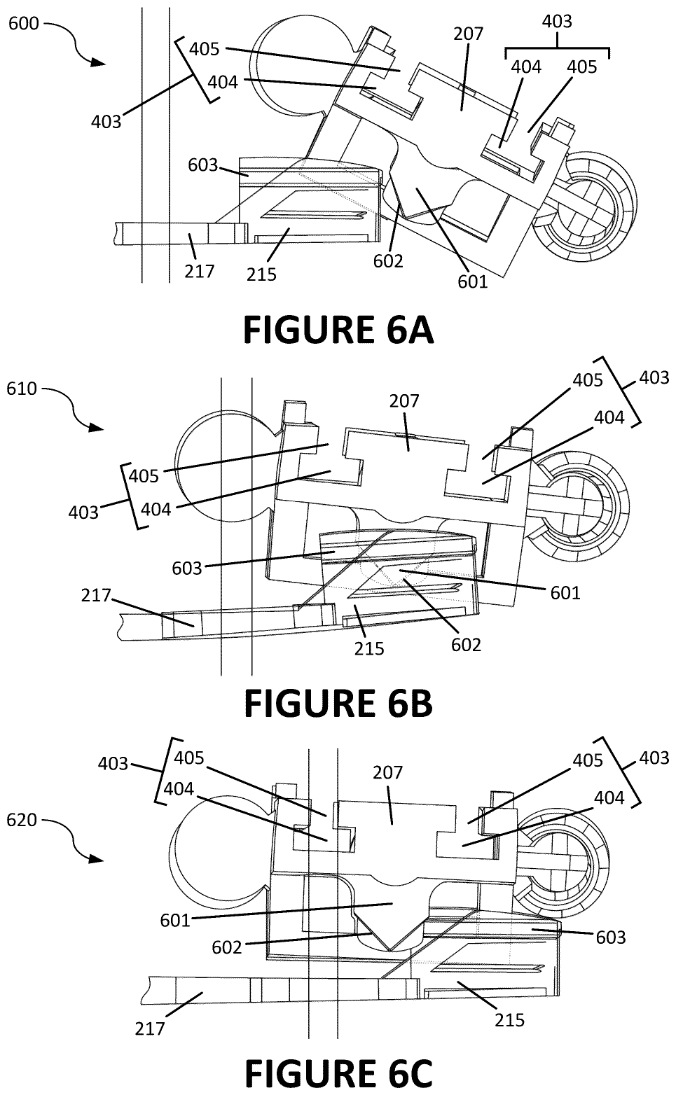

FIGS. 6A-C are cross-sectional side views showing the interaction of the cam post 207 with the wedge 215 as the drawer is moving from the open position to the closed position. FIG. 6A shows a cross-sectional side view 600 of the cam post 207 prior to its interaction with the wedge 215, FIG. 6B shows a cross-sectional side view 610 of the cam post 207 during its interaction with the wedge 215, and FIG. 6C shows a cross-sectional side view 620 of the cam post 207 after its interaction with the wedge 215.

As has been described, the cam post 207 comprises a pair of grooves 403, with each groove 403 comprising a lower section 404 and an upper section 405.

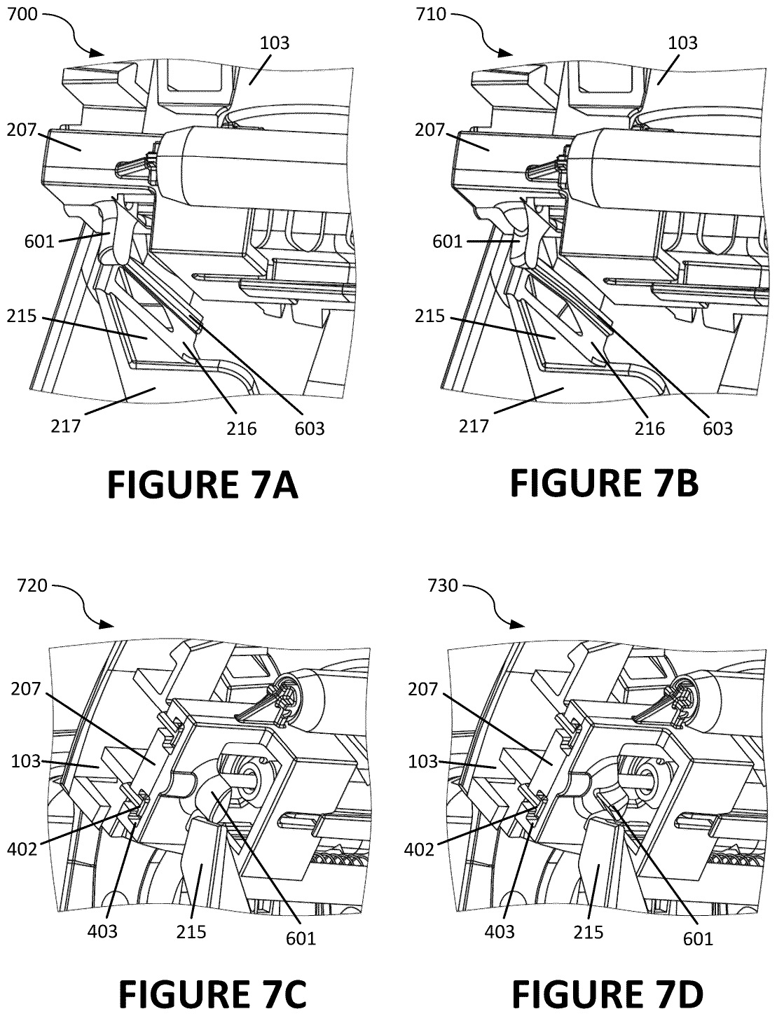

The cam post 207 may further comprise a block element 601 that protrudes downwards with respect to the longitudinal axis 106. The block element 601 may comprise an outer surface, which may be rounded or may be pointed. The outer surface of the block element 601 will be described in greater detail with respect to FIGS. 7A-D. Although rounded and pointed surfaces are described, it should be understood that other shaped surfaces are possible. The outer surface of the block element 601 may comprise a scraping edge 602 that protrudes out from the block element 601. The scraping edge 602 may be substantially V-shaped. This may help to increase the area of the inner side 216 of the wedge 215 over which the scraping edge 602 can remove residue.

As has been described, the wedge 215 is attached to a flexible arm 217. The inner side of the wedge 215 may comprise a scraping edge 603 which protrudes out from the inner side of the wedge 215. The scraping edge 603 may be substantially linear and may extend across the entirety of the inner side 216 of the wedge 215.

It should be understood that the cross-sectional views shown in FIGS. 6A-C are intended to be semi-transparent in order to better show the structure of the cam post 207 and the wedge 215. In FIGS. 6A-C, the cam post 207 is behind the wedge 215 and the scraping edge 603 of the wedge 215 is on the inner side of the wedge 215, which is the far side of the wedge 215 in these drawings.

The block element 601 of the cam post 207 is configured to interact with the wedge 215 which, as described, causes the perforating means to move from the resting position to the perforating position. The scraping edge 602 of the block element 601 is configured to remove residue from the portion of the main body of the inhaler with which it interacts-specifically, the wedge 215. This helps to prevent a build-up of residue from a capsule and helps to ensure that any disruption to air flow through the inhaler is minimised.

Similarly, the scraping edge 603 of the wedge 215 is configured to remove residue from the cam post 207 (specifically the block element 601) as the perforating means interacts with the wedge 215. This helps to prevent a build-up of residue from a capsule and helps to ensure that any disruption to air flow through the inhaler is minimised.

Starting at FIG. 6A, the interaction of the cam post 207 with the wedge 215 during a closing motion of the drawer will be described. The drawer starts in the open position and is then moved towards the closed position. FIG. 6A shows the cam post 207 and the wedge 215 prior to any interaction between the two elements.

As has been described, as the drawer is moved from the open position towards the closed position, the perforating means interact with a portion of the main body of the inhaler. More specifically, as is shown in FIG. 6B, the block element 601 of the cam post 207 interacts with the wedge 215. The cam post 207 slides along an inner side of the wedge 215, which causes the cam post 207 to move inwards along the transverse axis towards the center of the spin chamber. This movement is achieved by way of an interaction of the grooves 403 of the cam post 207 with the rails of the spin chamber.

During this stage, the scraping edge 602 of the block element is in contact with the wedge 215. The movement of the block element 601 along the inner side of the wedge 215 enables the scraping edge 602 to scrape off any residue that may be present on the inner side of the wedge 215. For example, there may be leftover residue from a previous capsule present on the wedge 215, and excessive build-up of residue may prevent the inhaler from working efficiently.

Simultaneously, the scraping edge 603 of the wedge 215 is in contact with the block element 601 during this stage. The movement of the block element 601 along the inner side of the wedge 215 enables the scraping edge 603 to scrape off any residue that may be present on the block element 601. For example, there may be leftover residue from a previous capsule present on the block element 601, and excessive build-up of residue may prevent the inhaler from working efficiently.

The scraping edge 603 of the wedge 215 may be in contact with the scraping edge 602 of the block element 601 during this process, or it may be in contact with another part of the block element 601. Similarly, the scraping edge 602 of the block element 601 may be in contact with the scraping edge 603 of the wedge 215 during this process, or it may be in contact with another part of the wedge 215.

The movement of the cam post 207 along the inner side of the wedge 215 may cause the wedge 215 to deviate slightly from its own rest position. As can be seen in FIG. 6B, the wedge 215 may be pulled upwards slightly. The arm 217 to which the wedge is attached is flexible, thus allowing movement of the wedge 215 and preventing any cracking or breaking.

FIG. 6C shows the cam post 207 after it has travelled along the entirety of the inner side of the wedge 215 and passed over the edge. In doing so, the cam post 207 moves back outwards along the transverse axis, meaning that the perforating means return to their resting position. The movement of the cam post 207 is achieved by way of an interaction of the grooves 403 of the cam post 207 with the rails of the spin chamber. The cam post 207 is then prevented from moving further outwards beyond the resting position by the supporting framework 205.

FIGS. 7A-D show perspective views of the cam post 207 interacting with the wedge 215. More specifically, FIG. 7A shows a perspective view 700 of a cam post 207 comprising a first type of block element 601, FIG. 7B shows a perspective view 710 of a cam post 207 comprising a second type of block element 601, FIG. 7C shows a different perspective view of a cam post 207 comprising the first type of block element 601 and FIG. 7D shows a different perspective view of a cam post 207 comprising the second type of block element 601.

Referring firstly to FIGS. 7A and 7B, the protrusion of the block element 601 downwards from the cam post 207 is shown. The block element 601 interacts with the wedge 215 as the drawer moves from the open position towards the closed position, as has been described. The protrusion of the scraping edge 603 from the inner side 216 of the wedge 215 is also shown.

The block element 601 may comprise a rounded surface, as shown in FIG. 7A. The rounded surface may comprise a scraping edge (not shown), as has been described with respect to FIGS. 6A-C. The use of a rounded surface may help to minimize the risk of the cam post getting stuck against the portion of the main body, since the rounded surface is smooth.

Alternatively the block element 601 may comprise a pointed surface, as shown in FIG. 7B. The pointed surface may comprise a scraping edge (not shown), as has been described with respect to FIGS. 6A-C. The use of a pointed surface may help to remove residue from the wedge 215.

FIG. 7C shows the block element 601 of FIG. 7A from a different angle, such that the curved surface is shown. FIG. 7D shows the block element 601 of FIG. 7B from a different angle, such that the pointed surface is shown.

FIG. 8 shows a cross-sectional side view 800 of the inhalation chimney 210 and the spin chamber 103 being held together in accordance with the present disclosure.

When the drawer is in the closed position, the inhalation chimney 210 is positioned directly above the spin chamber 103 with respect to the longitudinal axis 106. The spin chamber 103 may comprise a top surface 801 facing upwards with respect to the longitudinal axis 106. The top surface 801 may also be described as a top surface of the drawer, since the spin chamber 103 is located at the top of the drawer. The top surface 801 of the spin chamber 103 may be curved in a convex manner, as can be seen in FIG. 8.

The inhalation chimney 210 may comprise a bottom surface 802 facing downwards with respect to the longitudinal axis 106. The bottom surface 802 may be curved in concave manner corresponding to the curve of the top surface 801 of the spin chamber 103.

The top surface 801 and the bottom surface 802 are configured to be held together during inhalation, in order to define a chamber within which air can mix with the contents of a capsule inserted into the inhaler. This chamber may comprise the primary recess 104, the secondary recess 105 and a volume defined by the inhalation chimney 210. The curves of the two surfaces correspond to one another so that the spin chamber 103 and the inhalation chimney 210 may enclose the chamber.

When the drawer is in the closed position, the inhalation chimney 210 has been pulled downwards with respect to the longitudinal axis 106, as has been described. There may still be a small gap present between the top surface 801 and the bottom surface 802. As a user inhales through the mouthpiece of the inhaler, the negative pressure caused by the inhalation may cause the spin chamber 103 to move upwards slightly such that the top surface 801 and bottom surface 802 are in direct contact with each other. In this way, a seal may be formed between the two surfaces.

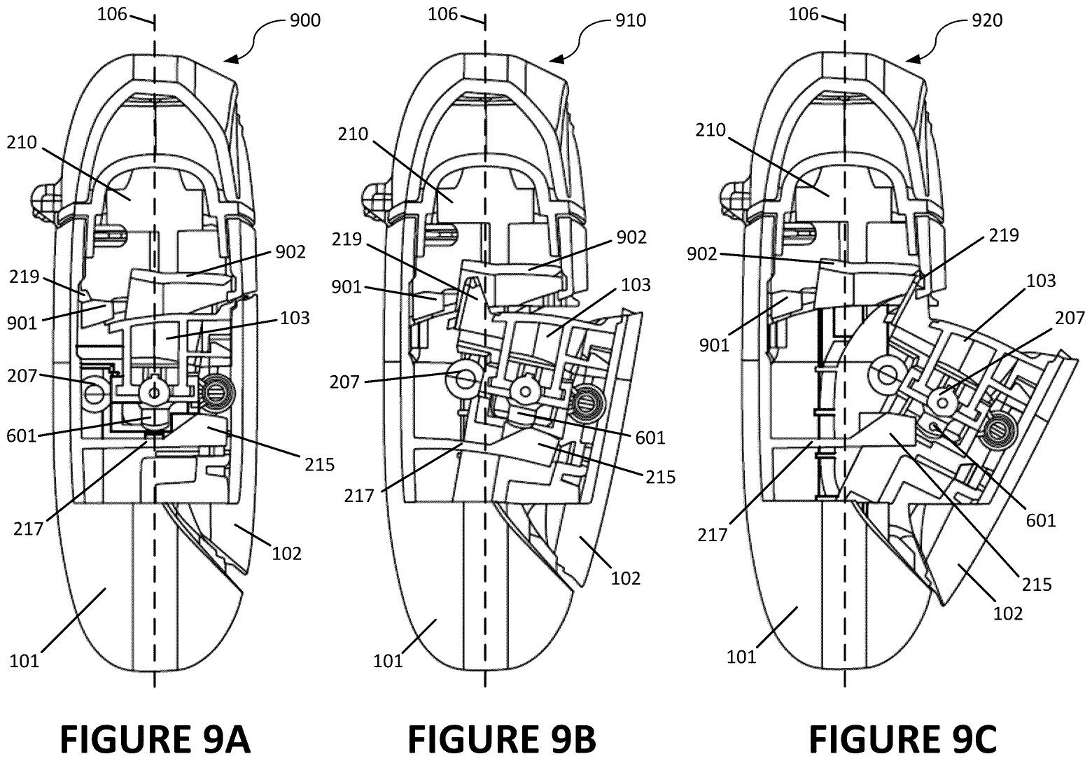

FIGS. 9A-C are a series of cross-sectional side views 900 of an inhaler showing the interaction of the cam post 207 with the wedge 215 as the drawer 102 is moved from a closed position to an open position. The inhaler may the same inhaler as described with reference to any of the previous figures.

The components and structure of the inhaler have already been discussed with reference to previous Figures.

Additionally, the inhalation chimney 210 of the inhaler comprises sealing ramps 901 positioned at the bottom of the inhalation chimney 210, on a side of the inhalation chimney 210 that is closest to the rear casing of the main body 101. The sealing ramps 210 may comprise a top surface. The inhalation chimney 210 also comprises protruding ribs 902 that protrude outwards along the transverse axis 218. Each protruding rib 902 may comprise a bottom surface facing downwards with respect to the longitudinal axis 106.

The functions of the drawer 102, perforating means, spin chamber 103 and inhalation chimney 210 have already been described with respect to the previous Figures.

The sealing ramps 901 are configured to receive the guide posts 219 of the spin chamber 103 as the drawer 102 moves from the open position to the closed position. The protruding ribs 902 are configured to interact with the guide posts 219 as the drawer 102 moves between the open position and the closed position. More specifically, the guide posts are configured to pass along the bottom surfaces of the protruding ribs 902 when moving towards the closed position and are configured to push the protruding ribs 902 upwards with respect to the longitudinal axis 106 when moving towards the open position.

FIG. 9A shows a cross-sectional side view 900 of the drawer 102 of the inhaler in the closed position, meaning that the drawer 102 has been fully closed into the main body 101 such that the components of the drawer 102 are enclosed within the inhaler. Having moved from the resting position to the perforating position during the closing of the drawer 102, the perforating means 204 have returned to the resting position.

The guide posts 219 have pulled the inhalation chimney 210 downwards along the longitudinal axis 106 and are held in place by the drawer retention clips (not shown).

FIG. 9B shows a cross-sectional side view 910 of the inhaler as the drawer is moving from the closed position towards the open position. As the user pulls on the drawer 102 to open it, the guide posts 219 push past the drawer retention clips and travel over the sealing ramps.

At the same time, the block element 601 of the cam post 207 travels over the top surface of the wedge 215. This movement causes the wedge 215, and the flexible arm 217 to which it is attached, to move downwards with respect to the longitudinal axis 106. The cam post 207 does not move along the transverse axis 218 as it passes over the wedge 215, meaning that the perforating means remain in the resting position as the drawer 102 moves towards the open position. The spring remains in its rest position during this movement.

FIG. 9C shows a cross-sectional side view 920 of the inhaler when the drawer 102 is in the open position. As the user continues to pull on the drawer 102 to open it, the guide posts 219 interact with the protruding ribs 902. More specifically, the guide posts 219 push the protruding ribs 902 upwards with respect to the longitudinal axis 106, which causes the inhalation chimney 210 to move upwards. This moves the inhalation chimney 210 away from the spin chamber 103, creating a gap which prevents the capsule from blocking the opening of the drawer 102. When in the open position, the guide posts 219 are prevented from moving beyond a particular point along the protruding ribs 902.

During this process, the cam post 207 passes over the top surface of the wedge 215 and eventually passes over a front edge of the wedge 215. Once this has occurred, the wedge 215 and the flexible arm 217 spring back upwards towards their normal rest position so that they are in the correct position for a subsequent opening of the drawer.

Once the drawer 102 is in the open position, the capsule can then be removed.

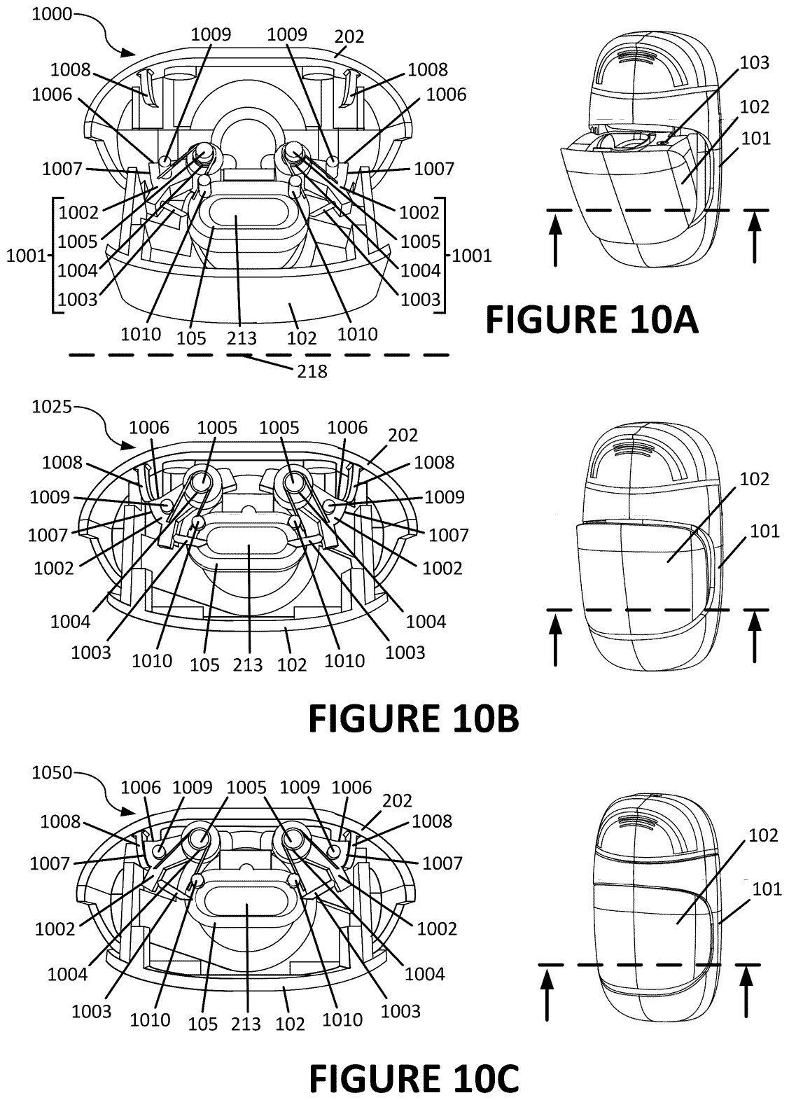

FIGS. 10A-C show a series of cross-sectional top views of an inhaler with an alternative form of the perforating means. The inhaler may be similar to any of the inhalers 100, 200 or 300, but with some differences with regard to the perforating means and the mechanism by which the perforating means perforate a capsule inserted into the inhaler. Specifically, in the embodiment of FIGS. 10A-C, the perforating means may be a rotatable perforating means 1001, rather than the perforating means 204 described with reference to the earlier Figures.

FIG. 10A is a cross-sectional bottom view 1000 of an inhaler showing the relative positions of the rotatable perforating means 1001 and the main body of the inhaler when the drawer is in an open position.

FIG. 10B is a cross-sectional bottom view 1025 of the inhaler showing the interaction of the rotatable perforating means 1001 with the main body of the inhaler as the drawer is being closed into the main body.

FIG. 10C is a cross-sectional bottom view 1050 of the inhaler showing the relative positions of the rotatable perforating means 1001 and the main body of the inhaler when the drawer is in a closed position.

In the inhaler shown in FIGS. 10A-C, the rotatable perforating means 1001 comprises a rotating element 1002, a needle 1003, a torsional spring 1004 and a pivot point 1005. The rotating element 1002 is coupled to a non-perforating end of the needle 1003. The needle 1003 extends away from the rotating element 1002 towards the center of the inhaler along the plane of the transverse axis 218. A perforating end of the needle 1003 is not directly connected to any other part of the inhaler.

The rotating element 1002 extends along its length from a first end to a second end. When viewed from the top or bottom, the rotating element 1002 has an irregular cross section and so comprises a number of surfaces. In particular, in the example shown in FIGS. 10A-C, the rotating element 1002 comprises a substantially flat upper surface, a substantially flat lower surface, a contact surface 1006 facing outwards from the center of the inhaler along the plane of the transverse axis 218, and a curved surface 1007 facing outwards towards the side of the inhaler along the plane of the transverse axis 218. The curved surface 1007 is at the first end of the rotating element 1002 and the contact surface 1006 is adjacent the curved surface 1007. The contact surface 1006 is substantially flat, while the curved surface 1007 is curved so as to provide a convex curve. The rotating element 1002 also comprises a first protrusion 1009 extending away from the upper surface of the rotating element 1002 along the longitudinal axis of the inhaler. The second end of the rotating element 1002 comprises a substantially flat surface configured to interact with the spin chamber 103 of the inhaler.

The pivot point 1005 is positioned at a suitable location along the length of the rotating element 1002, such that both ends of the rotating element 1002 may move during rotation of the rotating element 1002. In FIGS. 10A-C, the pivot point is closer to the second end of the rotating element 1002 than the first end. At the pivot point 1005, there is a pivot mechanism that enables the rotating element 1002 to rotate about the pivot point 1005 between a resting position and a perforating position. The perforating position is a position within the secondary recess 105 where the needle 1003 may perforate the capsule 213. When the rotating element 1002 is in the resting position, the needle 1003 is further away from the center of the spin chamber 103 than when the rotating element 1002 is in the perforating position. The pivot mechanism comprises suitable pivoting means on both the rotating element 1002 and the drawer 102. In the example shown in FIGS. 10A-C, the pivoting means on the drawer 102 are positioned on a bottom surface of the spin chamber 103.

The pivoting means comprises a protrusion extending downwards from the drawer 102 (which in this example is a pin) and a corresponding cavity (which in this example is a hole) extending through at least a portion of the rotating element 1002. Alternatively, the pivoting means may comprise a protrusion extending upwards from the bottom of the rotating element 1002 and a corresponding cavity extending through at least a portion of the drawer 102. It should be appreciated that any suitable means for enabling rotation of the rotating element 1002 with respect to the drawer 102 may be employed.

As can be seen in FIGS. 10A-C, the torsional spring 1004 is a wire that comprises a first linear section, a central helical section and a second linear section. The first linear section is substantially linear and extends from a first end of the torsional spring 1004 to the central helical section. The central helical section is a coil that wraps around the pivot point 1005 a number of times. The second linear section is substantially linear and extends from the central helical section to a second end of the torsional spring 1004.

The first end of the torsional spring 1004 is proximate a particular section of the rotating element 1002 such that a portion of the first linear section is in contact with the first protrusion 1009. This portion does not need to be coupled to the first protrusion 1009—it is sufficient that the two are simply in contact. In the example shown in FIGS. 10A-C, the first protrusion 1009 is substantially cylindrical, meaning that the contact between the first protrusion 1009 and the first linear section of the torsional spring 1004 is tangential.

The second end of the torsional spring 1004 is proximate a particular section of the drawer 102, such that a portion of the second linear section is in contact with a second protrusion 1010 extending outwards from the drawer 102. In FIGS. 10A-C, the second protrusion 1010 extends away from a bottom surface of the spin chamber 103 along the longitudinal axis of the inhaler. The portion of the second linear section does not need to be coupled to the second protrusion 1010—it is sufficient that the two are simply in contact. In the example shown in FIGS. 10A-C, the second protrusion 1010 is substantially cylindrical, meaning that the contact between the second protrusion 1010 and the second linear section of the torsional spring 1004 is tangential. The second protrusion 1010 may be substantially similar to the first protrusion 1009.

The torsional spring 1004 is in a rest state when the drawer 102 is in the open position and when the drawer 102 is in the closed position, but is gradually compressed as the drawer 102 moves from the open position to the closed position, as will be described in greater detail later on.

FIGS. 10A-C show the rotatable perforating means 1001 as comprising two sets of rotating elements 1002, needles 1003, torsional springs 1004 and pivot points 1005, with each set located on opposing sides of the spin chamber 103 with respect to the transverse axis 218, although the preceding paragraphs have so far described only one rotating element 1002, one needle 1003, one torsional spring 1004 and one pivot point 1005. It is to be understood that the inhaler may function with one rotating element 1002, one needle 1003, one torsional spring 1004 and one pivot point 1005, or with two rotating elements 1002, two needles 1003, two torsional springs 1004 and two pivot points 1005. The only requirements are that the rotatable perforating means 1001 comprises at least one rotating element 1002, at least one needle 1003, at least one torsional spring 1004 and at least one pivot point 1005. The number of rotating elements 1002, needles 1003, torsional springs 1004 and pivot points 1005 should be the same. In an embodiment, the rotatable perforating means 1001 comprises two rotating elements 1002, two needles 1003, two torsional springs 1004 and two pivot points 1005, as shown in FIGS. 10A-C, with each set of rotatable perforating means 1001 being located on opposing sides of the spin chamber 103.

The use of two needles 1003 results in two perforations of the capsule 213. This decreases the time required for the contents of the capsule 213 to be removed from the capsule 213 through inhalation, since there will be two holes created in opposing sides of the capsule 213. The needles 1003 are configured to perforate the capsule 213 at the same time. This helps to ensure an efficient and timely emptying of the capsule 213, since both holes will be created at the same time.

In the example of FIGS. 10A-C, the rear casing 202 of the inhaler comprises at least one actuating arm 1008 extending inwards from the inner surface of the rear casing 202 towards the center of the inhaler along an axis that is substantially perpendicular to the longitudinal axis 106 of the rear casing 202 and substantially perpendicular to the transverse axis 218. FIGS. 10A-C show an embodiment in which the rear casing 202 comprises two actuating arms 1008, both extending in substantially the same direction but positioned towards different sides of the inhaler. The number of actuating arms 1008 should be the same as the number of rotating elements 1002, needles 1003, torsional springs 1004 and pivot points 1005.

Each actuating arm 1008 extends towards its respective rotating element 1002, such that as the inhaler is closed, each actuating arm 1008 makes contact with the contact surface 1006 of its respective rotating element 1002. Each actuating arm 1008 is slightly curved so as to match the curve of the curved surface 1007 of its respective rotating element 1002.

The function of the rotatable perforating means 1001 is the same as the perforating means 204—namely, the rotatable perforating means 1001 are configured to perforate the capsule 213, thus releasing the contents of the capsule 213 and allowing them to mix with air so that they may be inhaled by a user. However, the rotatable perforating means 1001 are configured to achieve this in a different manner to the perforating means 204.

More specifically, the rotatable perforating means 1001 are configured to rotate inwards about the pivot point 1005 in the plane of the transverse axis 218 from the resting position to the perforating position as the drawer 102 moves from the open position to the closed position, due to contact with the actuating arms 1008, such that the needle 1003 swings inwards towards the capsule 213. When at the perforating position, which occurs shortly before the drawer 102 is in the closed position, the rotatable perforating means 1001 are configured to perforate the capsule 213 and then move back from the perforating position to the resting position. When the drawer 102 is in the closed position, the rotatable perforating means 1001 are in the resting position. As the drawer 102 moves from the closed position to the open position, the rotatable perforating means 1001 are configured to substantially remain in the resting position.

The rotatable perforating means 1001 are configured to interact with a portion of the main body of the inhaler as the drawer 102 moves between the open position and the closed position, which causes the rotatable perforating means 1001 to rotate from their resting position towards their perforating position. More specifically, in the embodiment of FIGS. 10A-C, the rotatable perforating means 1001 are configured to interact with the at least one actuating arm 1008, which is connected to the rear casing 202 of the inhaler. Similarly, the at least one actuating arm 1008 is configured to interact with the rotatable perforating means 1001 to assist in perforation of the capsule 213. The at least one actuating arm 1008 is configured to contact the contact surface 1006 of the rotating element 1002, thus providing a flat surface to flat surface contact interface. As the drawer 102 is closed further, the at least one actuating arm 1008 is then configured to provide a reactionary force to the rotating element 1002, thus causing it to rotate. The actuating arm 1008 is also configured to help keep the drawer 102 in the closed position once it has been closed—the curvature of the actuating arm 1008, which matches the curvature of the curved surface 1007 of the rotating element 1002, helps to prevent the drawer 102 from unintentionally reopening.

The rotating element 1002 is the component of the rotatable perforating means 1001 that is configured to interact with the actuating arm 1008. The rotating element 1002 is configured to rotate about the pivot point 1005 upon contact with the at least one actuating arm 1008 and move the needle 1003 towards the capsule to be perforated. The flat surface at the second end of the rotating element 1002 is configured to be in contact with the spin chamber 103 when the rotatable perforating means 1001 are in the resting position and thus prevents the rotating element 1002 from rotating further outwards beyond the resting position. The needle 1003, which is supported by the rotating element 1002, is configured to perforate the capsule. The torsional spring 1004 is configured to naturally keep the rotatable perforating means 1001 in the resting position, and to provide a resistive force as the rotating element 1002 rotates due to its interaction with the actuating arm 1008. This resistive force, which arises due to the movement of the first linear section of the torsional spring 1004 inwards during the movement of the rotating element 1002, helps to ensure that the other components of the rotatable perforating means 1001 remain in their required positions at all times.

The torsional spring 1004 is also configured to move the rotatable perforating means 1001 back to the resting position after the drawer 102 has been closed and the capsule has been pierced. Since at this point there is no contact between the contact surface 1006 of the rotating element 1002 and the actuating arm 1008, the torsional spring 1004 decompresses and spins the rotating element 1002 back to its resting position.

The first protrusion 1009 and the second protrusion 1010 are configured to hold the torsional spring 1004 in place. When the rotating element 1002 rotates inwards, the first protrusion 1009 is configured to push the first linear section of the torsional spring 1004 towards the second linear section of the torsional spring 1004. Since the spin chamber 103 does not rotate, the second protrusion 1010 is configured to hold the second linear section in place, such that the during rotation of the rotating element 1002, the first and second linear sections move closer together, thus compressing the torsional spring 1004 and providing the resistive force.

With specific reference now to FIG. 10A, the drawer 102 is shown to be in the open position. When in the open position, the spin chamber 103 is exposed so that a capsule (not shown) can be inserted into the secondary recess 105 of the spin chamber 103. The rotatable perforating means 1001 are in a resting position and cannot be moved any further away from the center of the spin chamber 103 along the transverse axis 218.

In the open position, the rotatable perforating means 1001 are arranged such that the needles 1003 are outside the secondary recess 105 and are unable to perforate the capsule located in the secondary recess 105. As has been described, the two needles 1003 are on opposite sides of the secondary recess 105 to one another, but in the open position, do not directly face each other due to the rotational positioning of the rotating elements 1002. The rotating elements 1002 are not in contact with the actuating arms 1008 while the drawer 102 is in the open position.

FIG. 10B shows the drawer 102 as it is moving from an open position towards a closed position. The movement of the drawer 102 causes the rotatable perforating means 1001 to interact with a portion of the main body of the inhaler. More specifically, the rotating elements 1002 interact with the actuating arms 1008. Each rotating element 1002 interacts with its respective actuating arm 1008 at the same time, and this interaction causes the rotating elements 1002 to rotate about their respective pivot points 1005. In the embodiment of FIGS. 10A-C, it can be seen that since the two rotating elements 1002 are substantially mirror images of each other and are positioned on opposite sides of the spin chamber 103 to each other, the rotating elements 1002 are configured to rotate in opposite directions to each other. A first of the two rotating elements 1002 (e.g. the rotating element 1002 on the left in FIGS. 10A-C) is configured to rotate anticlockwise upon interaction with its respective actuating arm 1008, while a second of the two rotating elements 1002 (e.g. the rotating element 1002 on the right in FIGS. 10A-C) is configured to rotate clockwise upon interaction with its respective actuating arm 1008.

This rotation causes the needles 1003 to move inwards towards the center of the spin chamber 103, against the biasing of their respective torsional springs 1004. The needles 1003 pass through the small apertures in the spin chamber 103 and enter the secondary recess 105. At this point, the rotatable perforating means 1001 are in the perforating position, and may perforate the capsule that is located in the secondary recess 105. Beneficially, this process takes place as the drawer 102 is being closed into the main body 101, without the need for the user to press a button or instigate perforation by any other means. In FIG. 10B, the rotatable perforating means 1001 are in the perforating position.

FIG. 10C shows the rotatable perforating means 1001 after perforation, when the drawer 102 is in the closed position. The continued movement of the drawer 102 towards the closed position causes the rotating elements 1002 to pass over the edges of their respective actuating arms 1008. Specifically, at this point, the actuating arms 1008 are no longer in contact with the contact surfaces 1006 of their respective rotating elements 1002. Once the rotating elements 1002 have passed over these edges, the torsional springs 1004, which were compressed during perforation, may decompress and cause the rotatable perforating means 1001 to return to the resting position. Specifically, the first linear section of the torsional spring 1004 pushes the first protrusion 1009, which causes the rotating element 1002 to rotate back towards its resting position. The rotatable perforating means 1001 are then held in place by the actuating arms 1008, which may also help to prevent them from rotating any further. At this point, the inhaler is closed, and may be used by a user to inhale the contents of the capsule.

To open the drawer 102 and remove the used capsule, a sufficient pulling force must be applied to the drawer 102 such that the rotating elements 1002 can push aside the actuating arms 1008, which are holding them in place. If sufficient force is applied, the drawer can be pulled open. The rotating elements 1002 may rotate slightly during this interaction, but not sufficiently to risk any jamming of the components. Once the rotating elements 1002 have passed the actuating arms 1008, the biasing of the torsional springs 1004 causes the rotating elements 1002 to rotate back to the resting position. From here on, the drawer 102 may be opened fully without the need for excessive force.

Various embodiments of the present disclosure include one or more of the following items:
1. An inhaler comprising: a main body; and a drawer configured to open out of and close into the main body between an open position and a closed position, the drawer comprising: a spin chamber comprising a primary recess configured to receive air to mix with contents of a capsule and a secondary recess configured to hold the capsule, wherein when the drawer is in the open position, the secondary recess is exposed to receive a new capsule therein or to withdraw a used capsule therefrom, and when the drawer is in the closed position, the capsule is enclosed within the inhaler; and perforating means configured to perforate the capsule, the perforating means configured to move away from a resting position toward a perforating position as the drawer moves into the main body from the open position towards the closed position, wherein set at the perforating position, the perforating means is positioned within the secondary recess such that the perforating means is configured to perforate the capsule, and wherein the perforating means is configured to move from the perforating position to the resting position as the drawer moves into the main body from the open position towards the closed position, such that when the drawer is in the closed position, the perforating means is in the resting position.
2. The inhaler of item 1, wherein the perforating means is configured to interact with a portion of the main body as the drawer moves into the main body from the open position towards the closed position, the interaction causing the perforating means to slide along the portion of the main body.
3. The inhaler of items 1 or 2, wherein the perforating means comprises at least one needle configured to transversely slide against the biasing of at least one spring, wherein the at least one spring is in a rest state when the perforating means are in the resting position.
4. The inhaler of item 3, wherein the at least one spring is coupled to a cam post, the cam post adapted to, as the drawer moves into the main body from the open position towards the closed position, slide along the portion of the main body and simultaneously compress the at least one spring, wherein the cam post is coupled to a non-perforating end of the needle.
5. The inhaler of item 4, wherein the cam post comprises a rounded surface, the rounded surface adapted to, as the drawer moves into the main body from the open position towards the closed position, slide along the portion of the main body.
6. The inhaler of item 4, wherein the cam post comprises a scraping edge for removing residue from the portion of the main body.
7. The inhaler of item 6, wherein the scraping edge is substantially V-shaped.
8. The inhaler of any of items 4 to 7, wherein the portion of the main body comprises a wedge connected to a flexible arm, and wherein the cam post is configured to slide along a side of the wedge as the drawer moves into the main body from the open position towards the closed position.
9. The inhaler of item 8, wherein the wedge comprises a scraping edge configured to remove residue from the cam post.
10. The inhaler of items 8 or 9, wherein when the drawer is proximate to the closed position, the cam post is configured to travel over an end of the wedge, thereby causing the at least one spring to decompress.
11. The inhaler of any of items 8 to 10, wherein the inhaler has a longitudinal axis extending from a top of the inhaler, through the main body and the drawer, to a bottom of the inhaler, and wherein as the drawer moves out of the main body from the closed position towards the open position: the cam post is configured to travel over a top surface of the wedge and cause the flexible arm to move downwards from a rest position along the longitudinal axis towards the bottom of the inhaler; and the at least one spring is configured to remain in the rest state.
12. The inhaler of item 11, wherein when the drawer is in the open position, the flexible arm is configured to return to the rest position.
13. The inhaler of any of items 3 to 12, wherein the at least one needle comprises a pair of opposing needles, each needle coupled to a respective at least one spring.
14. The inhaler of item 13, wherein the pair of opposing needles is configured to perforate the capsule at the same time.
15. The inhaler of item 14, wherein the pair of opposing needles is configured to perforate opposing ends of the capsule.
16. The inhaler of items 1 to 15, wherein the drawer is coupled to the inhaler by a hinge mechanism.
17. The inhaler of items 1 to 16, wherein at least a portion of the main body comprises wax-lubricated PBT.
18. The inhaler of items 1 to 17, further comprising at least one air inlet configured to allow air to flow through the inhaler and spin the capsule.

It will be appreciated from the discussion above that the embodiments shown in the Figures are merely exemplary, and include features which may be generalized, removed or replaced as described herein and as set out in the claims.

Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the disclosure, which is defined in the accompanying claims.

Where ranges are recited herein these are to be understood as disclosures of the limits of said range and any intermediate values between the two limits.

With reference to the drawings in general, it will be appreciated that schematic functional block diagrams are used to indicate functionality of systems and apparatus described herein. It will be appreciated however that the functionality need not be divided in this way and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

Method embodiments may be implemented using the apparatus described herein.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the disclosure, which is defined in the accompanying claims.

These claims are to be interpreted with due regard for equivalents.

1. An inhaler comprising:
a main body; and
a drawer configured to open out of and close into the main body between an open position and a closed position, the drawer comprising:
a spin chamber comprising a primary recess configured to receive air to mix with contents of a capsule and a secondary recess configured to hold the capsule, wherein when the drawer is in the open position, the secondary recess is exposed to receive a new capsule therein or to withdraw a used capsule therefrom, and when the drawer is in the closed position, the capsule is enclosed within the inhaler; and
perforating means configured to perforate the capsule, the perforating means configured to move away from a resting position toward a perforating position as the drawer moves into the main body from the open position towards the closed position, wherein set at the perforating position, the perforating means is positioned within the secondary recess such that the perforating means is configured to perforate the capsule, and
wherein the perforating means is configured to move from the perforating position to the resting position as the drawer moves into the main body from the open position towards the closed position, such that when the drawer is in the closed position, the perforating means is in the resting position.

2. The inhaler of claim 1, wherein the perforating means is configured to interact with a portion of the main body as the drawer moves into the main body from the open position towards the closed position, the interaction causing the perforating means to slide along the portion of the main body.

3. The inhaler of claim 1, wherein the perforating means comprises at least one needle configured to transversely slide against the biasing of at least one spring, wherein the at least one spring is in a rest state when the perforating means are in the resting position.

4. The inhaler of claim 3, wherein the at least one spring is coupled to a cam post, the cam post adapted to, as the drawer moves into the main body from the open position towards the closed position, slide along the portion of the main body and simultaneously compress the at least one spring, wherein the cam post is coupled to a non-perforating end of the needle.

5. The inhaler of claim 4, wherein the cam post comprises a rounded surface, the rounded surface adapted to, as the drawer moves into the main body from the open position towards the closed position, slide along the portion of the main body.

6. The inhaler of claim 4, wherein the cam post comprises a scraping edge for removing residue from the portion of the main body.

7. The inhaler of claim 6, wherein the scraping edge is substantially V-shaped.

8. The inhaler of claim 4, wherein the portion of the main body comprises a wedge connected to a flexible arm, and wherein the cam post is configured to slide along a side of the wedge as the drawer moves into the main body from the open position towards the closed position.

9. The inhaler of claim 8, wherein the wedge comprises a scraping edge configured to remove residue from the cam post.

10. The inhaler of claim 8, wherein when the drawer is proximate to the closed position, the cam post is configured to travel over an end of the wedge, thereby causing the at least one spring to decompress.

11. The inhaler of claim 8, wherein the inhaler has a longitudinal axis extending from a top of the inhaler, through the main body and the drawer, to a bottom of the inhaler, and wherein as the drawer moves out of the main body from the closed position towards the open position:
the cam post is configured to travel over a top surface of the wedge and cause the flexible arm to move downwards from a rest position along the longitudinal axis towards the bottom of the inhaler; and
the at least one spring is configured to remain in the rest state.

12. The inhaler of claim 11, wherein when the drawer is in the open position, the flexible arm is configured to return to the rest position.

13. The inhaler of claim 3, wherein the at least one needle comprises a pair of opposing needles, each needle is coupled to a respective at least one spring.

14. The inhaler of claim 13, wherein the pair of opposing needles is configured to perforate the capsule at the same time.

15. The inhaler of claim 14, wherein the pair of opposing needles is configured to perforate opposing ends of the capsule.

16. The inhaler of claim 1, wherein the drawer is coupled to the inhaler by a hinge mechanism.

17. The inhaler of claim 1, wherein at least a portion of the main body comprises wax-lubricated PBT.

18. The inhaler of claim 1, further comprising at least one air inlet configured to allow air to flow through the inhaler and spin the capsule.
